(19) United States
(12) Patent Application Publication
(10) Pub. No.: US 2008/0262229 A1
(43) Pub. Date: Oct. 23, 2008

(54) Diarylmethyl Piperazine Derivatives, Preparations Thereof and Uses Thereof

(75) Inventors: William Brown, Montreal (CA); Andrew Griffin, Montreal (CA); Andrea Penwell, Montreal (CA)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 11/816,656

(22) PCT Filed: Feb. 24, 2006
(86) PCT No.: PCT/SE2006/000248

§ 371 Date: Aug. 20, 2007

(30) Foreign Application Priority Data
Feb. 28, 2005 (SE) 0500454-4

(51) Int. Cl. C07D 241/04; A61K 31/4965; A61P 25/24

(52) U.S. Cl. 544/396; 514/255.04

(57) ABSTRACT

Compounds of formula:

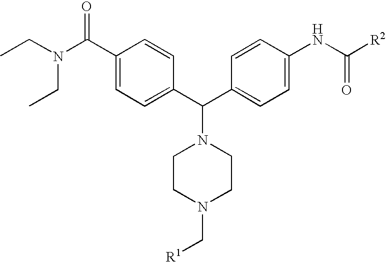

wherein R1 and R2 are as defined in the specification, as well as salts, enantiomers thereof and pharmaceutical compositions including the compounds are prepared. They are useful in therapy, in particular in the management of pain.

### FIELD OF THE INVENTION

The present invention is directed to novel compounds, to a process for their preparation, their use and pharmaceutical compositions comprising the novel compounds. The novel compounds are useful in therapy, and in particular for the treatment of pain, anxiety and depression.

### BACKGROUND OF THE INVENTION

The δ receptor has been identified as having a role in many bodily functions such as circulatory and pain systems. Ligands for the δ receptor may therefore find potential use as analgesics, and/or as antihypertensive agents. Ligands for the δ receptor have also been shown to possess immunomodulatory activities.

The identification of at least three different populations of opioid receptors (μ, δ and κ) is now well established and all three are apparent in both central and peripheral nervous systems of many species including man. Analgesia has been observed in various animal models when one or more of these receptors have been activated.

With few exceptions, currently available selective opioid δ ligands are peptidic in nature and are unsuitable for administration by systemic routes. One example of a non-peptidic δ-agonist is SNC80 (Bilsky E. J. et al., Journal of Pharmacology and Experimental Therapeutics, 273(1), pp. 359-366 (1995)).

Many δ agonist compounds that have been identified in the prior art have many disadvantages in that they suffer from poor pharmacokinetics and are not analgesic when administered by systemic routes. Also, it has been documented that many of these δ agonist compounds show significant convulsive effects when administered systemically.

U.S. Pat. No. 6,130,222 describes some δ-agonists.

However, there is still a need for improved δ-agonists.

### DESCRIPTION OF THE INVENTION

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures.

The term “Cₘ₋ₙ” or “Cₘ₋ₙ group” used alone or as a prefix, refers to any group having m to n carbon atoms.

The term “hydrocarbon” used alone or as a suffix or prefix, refers to any structure comprising only carbon and hydrogen atoms up to 14 carbon atoms.

The term “hydrocarbon radical” or “hydrocarbyl” used alone or as a suffix or prefix, refers to any structure as a result of removing one or more hydrogens from a hydrocarbon.

The term “alkyl” used alone or as a suffix or prefix, refers to a saturated monovalent straight or branched chain hydrocarbon radical comprising 1 to about 12 carbon atoms. Illustrative examples of alkyls include, but are not limited to, C₁₋₄alkyl groups, such as methyl, ethyl, propyl, isopropyl, 2-methyl-1-propyl, 2-methyl-2-propyl, butyl, isobutyl, t-butyl, and longer alkyl groups, such as pentyl, hexyl, heptyl, and octyl.

The term “heteroaryl” refers to a ring-containing monovalent radical having one or more heteroatoms, independently selected from N, O, P and S, as a part of the ring structure and including at least 3 and up to about 20 atoms in the ring(s), wherein the ring-containing radical has an aromatic character (e.g., 4n+2 delocalized electrons).

Exemplary heteroaryls are pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, triazinyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl.

The term “alkoxy” refers to radicals of the general formula —O—R, wherein R is selected from an alkyl. Exemplary alkoxy includes methoxy, ethoxy, propoxy, isopropoxy, butoxy, t-butoxy, and isobutoxy.

Halogen includes fluorine, chlorine, bromine and iodine.

“Halogenated,” used as a prefix of a group, means one or more hydrogens on the group are replaced with one or more halogens.

“RT” or “rt” means room temperature.

“—OTf” refers to —O—S(═O)₂—CF₃.

In one aspect, the invention provides a compound of formula I, a pharmaceutically acceptable salt thereof, diastereomers thereof, enantiomers thereof, and mixtures thereof:

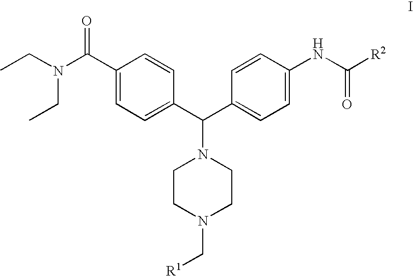

wherein

R¹ is selected from phenyl and C₃₋₅heteroaryl, wherein said phenyl and C₃₋₅heteroaryl are optionally substituted with one or more groups selected from C₁₋₄alkyl, C₁₋₄alkoxy, halogen, amino, —CF₃, and C₁₋₄acyl; and

R² is selected from —H, C₁₋₄alkyl and C₁₋₄alkoxy.

In one embodiment, the compounds of the present invention are represented by formula I, wherein

R¹ is selected from phenyl, pyridyl, furyl, thienyl, imidazolyl, triazolyl, pyrrolyl, thiazolyl, and pyridyl-N-oxide, wherein said phenyl, pyridyl, furyl, thienyl, imidazolyl, triazolyl, pyrrolyl, thiazolyl, and pyridyl-N-oxide are optionally substituted with one or more groups selected from halogen and C₁₋₄alkyl; and

R² is selected from —H, methyl, ethyl, methoxy and ethoxy.

In another embodiment, the compounds of the present invention are represented by formula I, wherein R¹ is selected from phenyl and pyridyl, wherein said phenyl and pyridyl are optionally substituted with one or more groups selected from methyl and fluoro; and

R² is selected from methyl and methoxy.

In a further embodiment, the compounds of the present invention are represented by formula I, wherein

R¹ is selected from phenyl, 2-fluorophenyl, 4-fluorophenyl, 2-pyridyl, 3-pyridyl, and 4-pyridyl; and

R² is selected from methyl and methoxy.

It will be understood that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of Formula I. The optically active forms of the compound of the invention may be prepared, for example, by chiral chromatographic separation of a racemate, by synthesis from optically active starting materials or by asymmetric synthesis based on the procedures described thereafter.

It will also be appreciated that certain compounds of the present invention may exist as geometrical isomers, for example E and Z isomers of alkenes. The present invention includes any geometrical isomer of a compound of Formula I. It will further be understood that the present invention encompasses tautomers of the compounds of the formula I.

It will also be understood that certain compounds of the present invention may exist in solvated, for example hydrated, as well as unsolvated forms. It will further be understood that the present invention encompasses all such solvated forms of the compounds of the formula I.

Within the scope of the invention are also salts of the compounds of the formula I. Generally, pharmaceutically acceptable salts of compounds of the present invention may be obtained using standard procedures well known in the art, for example by reacting a sufficiently basic compound, for example an alkyl amine with a suitable acid, for example, HCl or acetic acid, to afford a physiologically acceptable anion. It may also be possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

In one embodiment, the compound of formula I above may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

The novel compounds of the present invention are useful in therapy, especially for the treatment of various pain conditions such as chronic pain, neuropathic pain, acute pain, cancer pain, pain caused by rheumatoid arthritis, migraine, visceral pain etc. This list should however not be interpreted as exhaustive.

Compounds of the invention are useful as immunomodulators, especially for autoimmune diseases, such as arthritis, for skin grafts, organ transplants and similar surgical needs, for collagen diseases, various allergies, for use as anti-tumour agents and anti viral agents.

Compounds of the invention are useful in disease states where degeneration or dysfunction of opioid receptors is present or implicated in that paradigm. This may involve the use of isotopically labelled versions of the compounds of the invention in diagnostic techniques and imaging applications such as positron emission tomography (PET).

Compounds of the invention are useful for the treatment of diarrhoea, depression, anxiety and stress-related disorders such as post-traumatic stress disorders, panic disorder, generalized anxiety disorder, social phobia, and obsessive compulsive disorder, urinary incontinence, premature ejaculation, various mental illnesses, cough, lung oedema, various gastro-intestinal disorders, e.g. constipation, functional gastrointestinal disorders such as Irritable Bowel Syndrome and Functional Dyspepsia, Parkinson's disease and other motor disorders, traumatic brain injury, stroke, cardioprotection following miocardial infarction, spinal injury and drug addiction, including the treatment of alcohol, nicotine, opioid and other drug abuse and for disorders of the sympathetic nervous system for example hypertension.

Compounds of the invention are useful as an analgesic agent for use during general anaesthesia and monitored anaesthesia care. Combinations of agents with different properties are often used to achieve a balance of effects needed to maintain the anaesthetic state (e.g. amnesia, analgesia, muscle relaxation and sedation). Included in this combination are inhaled anaesthetics, hypnotics, anxiolytics, neuromuscular blockers and opioids.

Also within the scope of the invention is the use of any of the compounds according to the formula I above, for the manufacture of a medicament for the treatment of any of the conditions discussed above.

A further aspect of the invention is a method for the treatment of a subject suffering from any of the conditions discussed above, whereby an effective amount of a compound according to the formula I above, is administered to a patient in need of such treatment.

Thus, the invention provides a compound of formula I, or pharmaceutically acceptable salt or solvate thereof, as hereinbefore defined for use in therapy.

In a further aspect, the present invention provides the use of a compound of formula I, or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The term “therapeutic” and “therapeutically” should be construed accordingly. The term “therapy” within the context of the present invention further encompasses to administer an effective amount of a compound of the present invention, to mitigate either a pre-existing disease state, acute or chronic, or a recurring condition. This definition also encompasses prophylactic therapies for prevention of recurring conditions and continued therapy for chronic disorders.

The compounds of the present invention are useful in therapy, especially for the therapy of various pain conditions including, but not limited to: chronic pain, neuropathic pain, acute pain, back pain, cancer pain, and visceral pain.

In use for therapy in a warm-blooded animal such as a human, the compound of the invention may be administered in the form of a conventional pharmaceutical composition by any route including orally, intramuscularly, subcutaneously, topically, intranasally, intraperitoneally, intrathoracially, intravenously, epidurally, intrathecally, intracerebroventricularly and by injection into the joints.

In one embodiment of the invention, the route of administration may be orally, intravenously or intramuscularly.

The dosage will depend on the route of administration, the severity of the disease, age and weight of the patient and other factors normally considered by the attending physician, when determining the individual regimen and dosage level at the most appropriate for a particular patient.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substance, which may also act as diluents, flavoring agents, solubilizers, lubricants, suspending agents, binders, or table disintegrating agents; it can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture in then poured into convenient sized moulds and allowed to cool and solidify.

Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Depending on the mode of administration, the pharmaceutical composition will preferably include from 0.05% to 99% w (percent by weight), more preferably from 0.10 to 50% w, of the compound of the invention, all percentages by weight being based on total composition.

A therapeutically effective amount for the practice of the present invention may be determined, by the use of known criteria including the age, weight and response of the individual patient, and interpreted within the context of the disease which is being treated or which is being prevented, by one of ordinary skills in the art.

Within the scope of the invention is the use of any compound of formula I as defined above for the manufacture of a medicament.

Also within the scope of the invention is the use of any compound of formula I for the manufacture of a medicament for the therapy of pain.

Additionally provided is the use of any compound according to Formula I for the manufacture of a medicament for the therapy of various pain conditions including, but not limited to: chronic pain, neuropathic pain, acute pain, back pain, cancer pain, and visceral pain.

A further aspect of the invention is a method for therapy of a subject suffering from any of the conditions discussed above, whereby an effective amount of a compound according to the formula I above, is administered to a patient in need of such therapy.

Additionally, there is provided a pharmaceutical composition comprising a compound of Formula I, or a pharmaceutically acceptable salt thereof, in association with a pharmaceutically acceptable carrier.

Particularly, there is provided a pharmaceutical composition comprising a compound of Formula I, or a pharmaceutically acceptable salt thereof, in association with a pharmaceutically acceptable carrier for therapy, more particularly for therapy of pain.

Further, there is provided a pharmaceutical composition comprising a compound of Formula I, or a pharmaceutically acceptable salt thereof, in association with a pharmaceutically acceptable carrier use in any of the conditions discussed above.

In a further aspect, the present invention provides a method of preparing a compound of formula I.

In one embodiment, the present invention provides a process for preparing a compound of formula I, comprising:

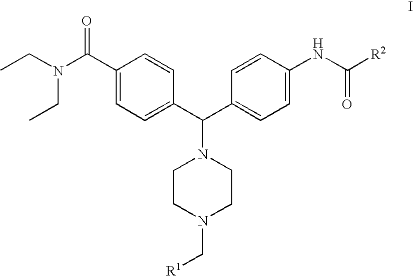

reacting a compound of formula II with R²—C(═O)—NH₂,

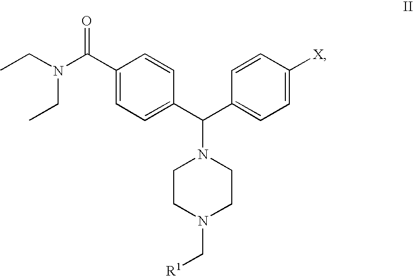

wherein X is selected from halogen and —OTf; and

R¹ and R² are as defined above.

Another embodiment of the invention provides a process of making a compound of formula I described above, wherein the step of reacting a compound of formula II with R²—C(═O)—NH₂ may be carried out in the presence of a reagent selected from xantphos, Pd₂(dba)₃, and Cs₂CO₃.

In a further embodiment, the present invention provides a process for preparing a compound of formula I, comprising:

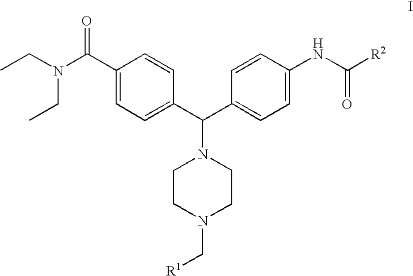

reacting a compound of formula III with R¹—CHO or R¹—CH₂X:

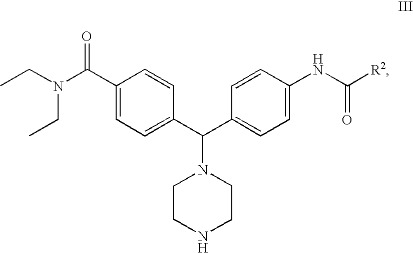

wherein X is selected from halogen and —OTf, and

R¹ and R² are as defined above.

An even further embodiment of the invention provides a process of preparing a compound of formula I, wherein the step of reacting a compound of formula III with R¹—CHO is carried out in the presence a reducing agent such as NaBH(OAc)₃.

Particularly, the compounds of the present invention and intermediates used for the preparation thereof can be prepared according to the synthetic routes as exemplified in Schemes 1-3.

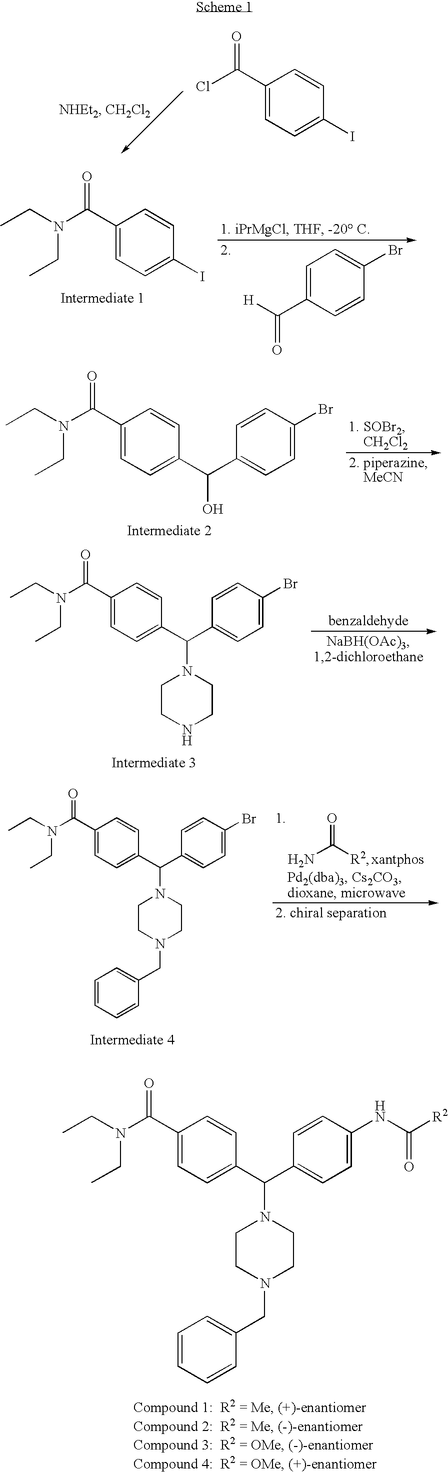

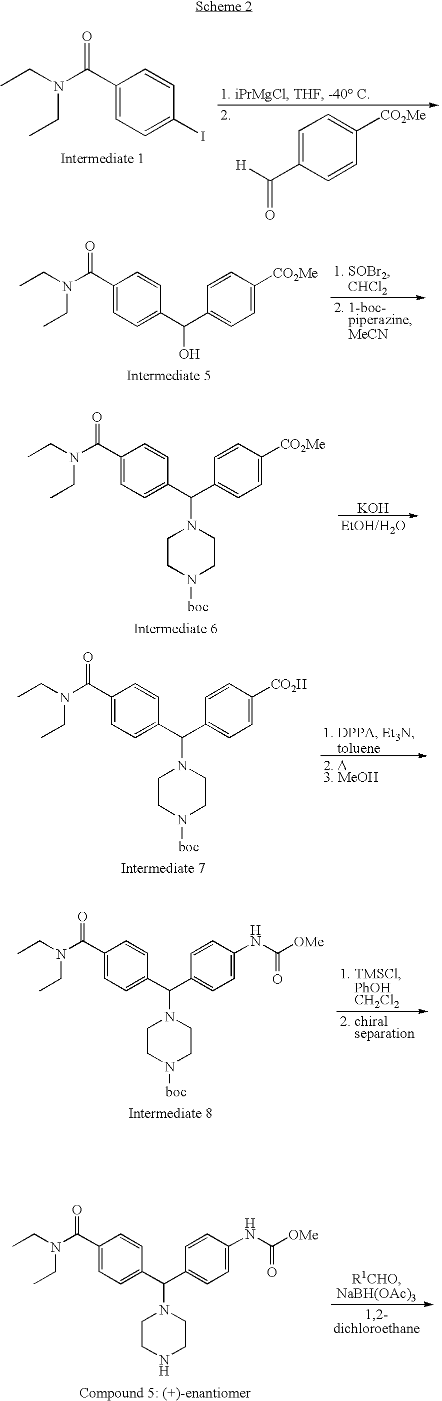

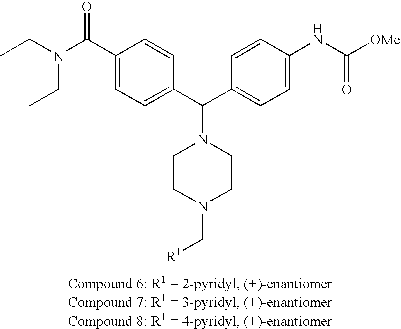

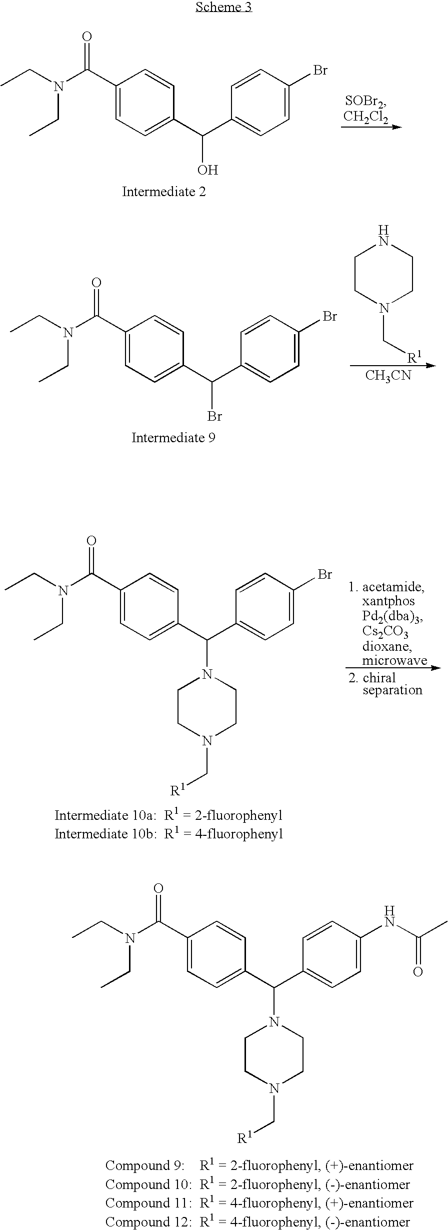

### Biological Evaluation

The compounds of the invention are found to be active towards δ receptors in warm-blooded animal, e.g., human. Particularly the compounds of the invention are found to be effective δ receptor ligands. In vitro assays, infra, demonstrate these surprising activities, especially with regard to agonist potency and efficacy as demonstrated in the rat brain functional assay and/or the human δ receptor functional assay. This feature may be related to in vivo activity and may not be linearly correlated with binding affinity. In these in vitro assays, a compound is tested for their activity toward δ receptors and IC₅₀ is obtained to determine the selective activity for a particular compound towards δ receptors. In the current context, IC₅₀ generally refers to the concentration of the compound at which 50% displacement of a standard radioactive δ receptor ligand has been observed.

The activities of the compound towards κ and μ receptors are also measured in a similar assay.

### In Vitro Model

### Cell Culture

Human 293S cells expressing cloned human κ, δ and μ receptors and neomycin resistance are grown in suspension at 37° C. and 5% CO₂ in shaker flasks containing calcium-free DMEM10% FBS, 5% BCS, 0.1% Pluronic F-69, and 600 μg/ml geneticin.

Rat brains are weighed and rinsed in ice-cold PBS (containing 2.5 mM EDTA, pH 7.4). The brains are homogenized with a polytron for 30 sec (rat) in ice-cold lysis buffer (50 mM Tris, pH 7.0, 2.5 mM EDTA, with phenylmethylsulfonyl fluoride added just prior use to 0.5 MmM from a 0.5M stock in DMSO:ethanol).

### Membrane Preparation

Cells are pelleted and resuspended in lysis buffer (50 mM Tris, pH 7.0, 2.5 mM EDTA, with PMSF added just prior to use to 0.1 mM from a 0.1 M stock in ethanol), incubated on ice for 15 min, then homogenized with a polytron for 30 sec. The suspension is spun at 1000 g (max) for 10 min at 4° C. The supernatant is saved on ice and the pellets resuspended and spun as before. The supernatants from both spins are combined and spun at 46,000 g (max) for 30 min. The pellets are resuspended in cold Tris buffer (50 mM Tris/CI, pH 7.0) and spun again. The final pellets are resuspended in membrane buffer (50 mM Tris, 0.32 M sucrose, pH 7.0). Aliquots (1 ml) in polypropylene tubes are frozen in dry ice/ethanol and stored at −70° C. until use. The protein concentrations are determined by a modified Lowry assay with sodium dodecyl sulfate.

### Binding Assays

Membranes are thawed at 37° C., cooled on ice, passed 3 times through a 25-gauge needle, and diluted into binding buffer (50 mM Tris, 3 mM MgCl₂, 1 mg/ml BSA (Sigma A-7888), pH 7.4, which is stored at 4° C. after filtration through a 0.22 m filter, and to which has been freshly added 5 μg/ml aprotinin, 10 μM bestatin, 10 μM diprotin A, no DTT). Aliquots of 100 μl are added to iced 12×75 mm polypropylene tubes containing 100 μl of the appropriate radioligand and 100 μl of test compound at various concentrations. Total (TB) and nonspecific (NS) binding are determined in the absence and presence of 10 μM naloxone respectively. The tubes are vortexed and incubated at 25° C. for 60-75 min, after which time the contents are rapidly vacuum-filtered and washed with about 12 ml/tube iced wash buffer (50 mM Tris, pH 7.0, 3 mM MgCl₂) through GF/B filters (Whatman) presoaked for at least 2 h in 0.1% polyethyleneimine. The radioactivity (dpm) retained on the filters is measured with a beta counter after soaking the filters for at least 12 h in minivials containing 6-7 ml scintillation fluid. If the assay is set up in 96-place deep well plates, the filtration is over 96-place PEI-soaked unifilters, which are washed with 3×1 ml wash buffer, and dried in an oven at 55° C. for 2 h. The filter plates are counted in a TopCount (Packard) after adding 50 μl MS-20 scintillation fluid/well.

### Functional Assays

The agonist activity of the compounds is measured by determining the degree to which the compounds receptor complex activates the binding of GTP to G-proteins to which the receptors are coupled. In the GTP binding assay, GTP[γ]³⁵S is combined with test compounds and membranes from HEK-293S cells expressing the cloned human opioid receptors or from homogenised rat and mouse brain. Agonists stimulate GTP[γ]³⁵S binding in these membranes. The EC₅₀ and Eₘₐₓ values of compounds are determined from dose-response curves. Right shifts of the dose response curve by the delta antagonist naltrindole are performed to verify that agonist activity is mediated through delta receptors. The Eₘₐₓ values were determined in relation to the standard δ agonist SNC80, i.e., higher than 100% is a compound that have better efficacy than SNC80.

### Procedure for Rat Brain GTP

Rat brain membranes are thawed at 37° C., passed 3 times through a 25-gauge blunt-end needle and diluted in the GTPγS binding (50 mM Hepes, 20 mM NaOH, 100 mM NaCl, 1 mM EDTA, 5 mM MgCl₂, pH 7.4, Add fresh: 1 mM DTT, 0.1% BSA). 120 μM GDP final is added membranes dilutions. The EC50 and Emax of compounds are evaluated from 10-point dose-response curves done in 300 μl with the appropriate amount of membrane protein (20 μg/well) and 100000-130000 dpm of GTPγ³⁵S per well (0.11-0.14 nM). The basal and maximal stimulated binding are determined in absence and presence of 3 μM SNC-80

### Data Analysis

The specific binding (SB) was calculated as TB−NS, and the SB in the presence of various test compounds was expressed as percentage of control SB. Values of IC₅₀ and Hill coefficient (n_{H}) for ligands in displacing specifically bound radioligand were calculated from logit plots or curve fitting programs such as Ligand, GraphPad Prism, SigmaPlot, or ReceptorFit. Values of Kᵢ were calculated from the Cheng-Prussoff equation. Mean ±S.E.M. values of IC₅₀, Kᵢ and n_{H} were reported for ligands tested in at least three displacement curves.

Based on the above testing protocols, we find that the compounds of the present invention are active toward human δ receptors.

### Receptor Saturation Experiments

Radioligand K_{δ} values are determined by performing the binding assays on cell membranes with the appropriate radioligands at concentrations ranging from 0.2 to 5 times the estimated K_{δ} (up to 10 times if amounts of radioligand required are feasible). The specific radioligand binding is expressed as pmole/mg membrane protein. Values of K_{δ} and Bₘₐₓ from individual experiments are obtained from nonlinear fits of specifically bound (B) vs. nM free (F) radioligand from individual according to a one-site model.

### Determination of Mechano-Allodynia Using Von Frey Testing

Testing is performed between 08:00 and 16:00 h using the method described by Chaplan et al. (1994). Rats are placed in Plexiglas cages on top of a wire mesh bottom which allows access to the paw, and are left to habituate for 10-15 min. The area tested is the mid-plantar left hind paw, avoiding the less sensitive foot pads. The paw is touched with a series of 8 Von Frey hairs with logarithmically incremental stiffness (0.41, 0.69, 1.20, 2.04, 3.63, 5.50, 8.51, and 15.14 grams; Stoelting, Ill., USA). The von Frey hair is applied from underneath the mesh floor perpendicular to the plantar surface with sufficient force to cause a slight buckling against the paw, and held for approximately 6-8 seconds. A positive response is noted if the paw is sharply withdrawn. Flinching immediately upon removal of the hair is also considered a positive response. Ambulation is considered an ambiguous response, and in such cases the stimulus is repeated.

### Testing Protocol

The animals are tested on postoperative day 1 for the FCA-treated group. The 50% withdrawal threshold is determined using the up-down method of Dixon (1980). Testing is started with the 2.04 g hair, in the middle of the series. Stimuli are always presented in a consecutive way, whether ascending or descending. In the absence of a paw withdrawal response to the initially selected hair, a stronger stimulus is presented; in the event of paw withdrawal, the next weaker stimulus is chosen. Optimal threshold calculation by this method requires 6 responses in the immediate vicinity of the 50% threshold, and counting of these 6 responses begins when the first change in response occurs, e.g. the threshold is first crossed. In cases where thresholds fall outside the range of stimuli, values of 15.14 (normal sensitivity) or 0.41 (maximally allodynic) are respectively assigned. The resulting pattern of positive and negative responses is tabulated using the convention, X=no withdrawal; O=withdrawal, and the 50% withdrawal threshold is interpolated using the formula:

50% g threshold=10^{(Xf+kδ)}/10,000

where Xf=value of the last von Frey hair used (log units); k=tabular value (from Chaplan et al. (1994)) for the pattern of positive/negative responses; and δ=mean difference between stimuli (log units). Here δ=0.224.

Von Frey thresholds are converted to percent of maximum possible effect (% MPE), according to Chaplan et al. 1994. The following equation is used to compute % MPE:

$$\% M P E = \frac{\begin{matrix}Drug treated threshold ( g ) - \\ allodynia threshold ( g ) \times 100\end{matrix}}{Control threshold ( g ) - allodynia threshold ( g )}$$

### Administration of Test Substance

Rats are injected (subcutaneously, intraperitoneally, intravenously or orally) with a test substance prior to von Frey testing, the time between administration of test compound and the von Frey test varies depending upon the nature of the test compound.

### Writhing Test

Acetic acid will bring abdominal contractions when administered intraperitoneally in mice. These will then extend their body in a typical pattern. When analgesic drugs are administered, this described movement is less frequently observed and the drug selected as a potential good candidate.

A complete and typical Writhing reflex is considered only when the following elements are present: the animal is not in movement; the lower back is slightly depressed; the plantar aspect of both paws is observable. In this assay, compounds of the present invention demonstrate significant inhibition of writhing responses after oral dosing of 1-100 μmol/kg.

### (i) Solutions Preparation

Acetic acid (AcOH): 120 μL of Acetic Acid is added to 19.88 ml of distilled water in order to obtain a final volume of 20 ml with a final concentration of 0.6% AcOH. The solution is then mixed (vortex) and ready for injection.
Compound (drug): Each compound is prepared and dissolved in the most suitable vehicle according to standard procedures.

### (ii) Solutions Administration

The compound (drug) is administered orally, intraperitoneally (i.p.), subcutaneously (s.c.) or intravenously (i.v.)) at 10 ml/kg (considering the average mice body weight) 20, 30 or 40 minutes (according to the class of compound and its characteristics) prior to testing. When the compound is delivered centrally: Intraventricularly (i.c.v.) or intrathecally (i.t.) a volume of 5 μL is administered.

The AcOH is administered intraperitoneally (i.p.) in two sites at 10 ml/log (considering the average mice body weight) immediately prior to testing.

(iii) Testing

The animal (mouse) is observed for a period of 20 minutes and the number of occasions (Writhing reflex) noted and compiled at the end of the experiment. Mice are kept in individual “shoe box” cages with contact bedding. A total of 4 mice are usually observed at the same time: one control and three doses of drug.

For the anxiety and anxiety-like indications, efficacy has been established in the geller-seifter conflict test in the rat.

For the functional gastrointestinal disorder indication, efficacy can be established in the assay described by Coutinho S V et al, in American Journal of Physiology—Gastrointestinal & Liver Physiology. 282(2):G307-16, 2002 February, in the rat.

### Additional In Vivo Testing Protocols

### Subjects and Housing

Naïve male Sprague Dawley rats (175-200 g) are housed in groups of 5 in a temperature controlled room (22° C., 40-70% humidity, 12-h light/dark). Experiments are performed during the light phase of the cycle. Animals have food and water ad libitum and are sacrificed immediately after data acquisition.

### Sample

Compound (Drug) testing includes groups of rats that do not receive any treatment and others that are treated with E. coli lipopolysaccharide (LPS). For the LPS-treated experiment, four groups are injected with LPS, one of the four groups is then vehicle-treated whilst the other three groups are injected with the drug and its vehicle. A second set of experiments is conducted involving five groups of rats; all of which receive no LPS treatment. The naïve group receives no compound (drug) or vehicle; the other four groups are treated with vehicle with or without drug. These are performed to determine anxiolytic or sedative effects of drugs which can contribute to a reduction in USV.

### Administration of LPS

Rats are allowed to habituate in the experimental laboratory for 15-20 min prior to treatment. Inflammation is induced by administration of LPS (endotoxin of gram-negative E. coli bacteria serotype 0111:B4, Sigma). LPS (2.4 μg) is injected intracerebro-ventricularly (i.c.v.), in a volume of 101, using standard stereotaxic surgical techniques under isoflurane anaesthesia. The skin between the ears is pushed rostrally and a longitudinal incision of about 1 cm is made to expose the skull surface. The puncture site is determined by the coordinates: 0.8 mm posterior to the bregma, 1.5 mm lateral (left) to the lambda (sagittal suture), and 5 mm below the surface of the skull (vertical) in the lateral ventricle. LPS is injected via a sterile stainless steel needle (26-G ⅜) of 5 mm long attached to a 100-μl Hamilton syringe by polyethylene tubing (PE20; 10-15 cm). A 4 mm stopper made from a cut needle (20-G) is placed over and secured to the 26-G needle by silicone glue to create the desired 5 mm depth.

Following the injection of LPS, the needle remains in place for an additional 10 s to allow diffusion of the compound, then is removed. The incision is closed, and the rat is returned to its original cage and allowed to rest for a minimum of 3.5 h prior to testing.

### Experimental Setup for Air-Puff Stimulation

The rats remain in the experimental laboratory following LPS injection and compound (drug) administration. At the time of testing all rats are removed and placed outside the laboratory. One rat at a time is brought into the testing laboratory and placed in a clear box (9×9×18 cm) which is then placed in a sound-attenuating ventilated cubicle measuring 62(w)×35(d)×46(h) cm (BRS/LVE, Div. Tech-Serv Inc). The delivery of air-puffs, through an air output nozzle of 0.32 cm, is controlled by a system (AirStim, San Diego Instruments) capable of delivering puffs of air of fixed duration (0.2 s) and fixed intensity with a frequency of 1 puff per 10 s. A maximum of 10 puffs are administered, or until vocalisation starts, which ever comes first. The first air puff marks the start of recording.

### Experimental Setup for and Ultrasound Recording

The vocalisations are recorded for 10 minutes using microphones (G.R.A.S. sound and vibrations, Vedbaek, Denmark) placed inside each cubicle and controlled by LMS (LMS CADA-X 3.5B, Data Acquisition Monitor, Troy, Mich.) software. The frequencies between 0 and 32000 Hz are recorded, saved and analysed by the same software (LMS CADA-X 3.5B, Time Data Processing Monitor and UPA (User Programming and Analysis)).

### Compounds (Drugs)

All compounds (drugs) are pH-adjusted between 6.5 and 7.5 and administered at a volume of 4 ml/kg. Following compound (drug) administration, animals are returned to their original cages until time of testing.

### Analysis

The recording is run through a series of statistical and Fourier analyses to filter (between 20-24 kHz) and to calculate the parameters of interest. The data are expressed as the mean ±SEM. Statistical significance is assessed using T-test for comparison between naive and LPS-treated rats, and one way ANOVA followed by Dunnett's multiple comparison test (post-hoc) for drug effectiveness. A difference between groups is considered significant with a minimum p value of ≦0.05. Experiments are repeated a minimum of two times.

### EXAMPLES

The invention will further be described in more detail by the following Examples which describe methods whereby compounds of the present invention may be prepared, purified, analyzed and biologically tested, and which are not to be construed as limiting the invention.

### INTERMEDIATE 1: 4-Iodo-N,N-diethylbenzamide

To a mixture of 4-iodobenzoyl chloride (100 g) in dichloromethane (600 mL) was added diethylamine (80 mL) dropwise at 0° C. After the addition was complete, the resulting reaction mixture was warmed to room temperature and stirred for 24 h. The mixture was then washed with saturated ammonium chloride. The organic extract was dried (Na₂SO₄), filtered and concentrated. The residue was recrystallized from hot ethanol/water (4:3) to give 95 g of INTERMEDIATE 1 as colourless needles.

### INTERMEDIATE 2: 4-[(4-bromophenyl)(hydroxy)methyl]-N,N-diethylbenzamide

To a mixture of THF (66 mL) and methanol (0.7 mL) cooled to −20° C. was added isopropylmagnesium chloride (2.0 M solution in diethyl ether) (24.7 mL, 49.5 mmol). The solution was allowed to reach room temperature and stirred for 20 min. The reaction was then cooled to 0° C. and a solution of INTERMEDIATE 1 (10.0 g, 33.0 mmol) in dry THF (40 mL) was slowly added over 30 min. The mixture was allowed to reach room temperature, stirred for 45 min, and then cooled again to 0° C.

A solution of 4-bromobenzaldehyde (6.23 g, 33.7 mmol) in dry THF (50 mL) was added dropwise over 30 min. The reaction was then warmed to room temperature and stirred overnight. The next day, saturated aqueous ammonium chloride was added. The mixture was stirred for 30 min and then extracted with two portions of dichloromethane. The combined organic extracts were dried (Na₂SO₄), filtered and concentrated. Column chromatography, eluting with 50% ethyl acetate in hexanes gave INTERMEDIATE 2 (8.46 g; 71% yield). Purity (HPLC-215 nm): >87%; ¹H NMR (400 MHz, CDCl₃) δ_{H} 0.95-1.34 (m, 6H), 3.11-3.33 (br s, 2H), 3.38-3.62 (br s, 2H), 5.75 (s, 1H), 7.22 (d, J=8.79 Hz, 2H), 7.26-7.35 (m, 4H), 7.43 (d, J=8.59 Hz, 2H).

### INTERMEDIATE 3: 4-[(4-bromophenyl)(piperazin-1-Yl)methyl]-N,N-diethylbenzamide

To a solution of INTERMEDIATE 2 (8.41 g, 23.2 mmol) in dichloromethane (75 mL) was slowly added thionyl bromide (2.16 mL, 27.9 mmol). The reaction was stirred for 6 h at room temperature. The solution was washed with saturated aqueous sodium bicarbonate and the organic layer was separated. The aqueous layer was washed with three portions of dichloromethane and the combined organic extracts were dried (Na₂SO₄), filtered and concentrated.

The crude benzyl bromide was dissolved in acetonitrile (230 mL) and piperazine (6.40 g, 74.3 mmol) was added. After heating the reaction for 2 h at 65° C., the reaction mixture was cooled to room temperature and washed with saturated ammonium chloride/ethyl acetate and the organic layer was separated. The aqueous layer was extracted with three portions of ethyl acetate and the combined organic extracts were dried (Na₂SO₄), filtered and concentrated. The residue was purified by flash chromatography, eluting with 2% methanol in dichloromethane to give INTERMEDIATE 3 (7.13 g, 71%) as a light yellow solid. This material was used directly in the next step.

### INTERMEDIATE 4: 4-[(4-benzylpiperazin-1-yl)(4-bromophenyl)methyl]-N,N-diethylbenzamide

To a solution of INTERMEDIATE 3 (1.39 g, 3.23 mmol) in 1,2-dichloroethane (50 mL) was added benzaldehyde (0.53 mL, 5.2 mmol) and sodium triacetoxyborohydride (1.16 g, 5.49 mmol). The reaction was stirred overnight at room temperature under nitrogen. The mixture was washed with saturated aqueous sodium bicarbonate and the aqueous layer was extracted with two portions of dichloromethane. The combined organic extracts were dried (MgSO₄), filtered and concentrated. The residue was purified by flash chromatography, eluting with 3% methanol in dichloromethane to give INTERMEDIATE 4 (1.46 g, 87%) as a colourless foam. ¹H NMR (400 MHz, CDCl₃) δ_{H} 1.04-1.14 (m, 3H), 1.17-1.27 (m, 3H), 1.64-1.74 (m, 2H), 2.30-2.56 (m, 6H), 3.17-3.29 (m, 2H), 3.45-3.59 (m, 4H), 4.21 (s, 1H), 7.21-7.32 (m, 9H), 7.35-7.42 (m, 4H). M.S. (calcd): 520.19 (MH⁺), M.S (found): 520.06 (MH⁺).

### INTERMEDIATE 5: methyl 4-[{4-[(diethylamino)carbonyl]phenyl}(hydroxy)methyl]benzoate

A solution of INTERMEDIATE 1 (10.0 g, 33.0 mmol) in THF (80 mL) was added dropwise to a solution of isopropylmagnesium chloride (18.2 mL, 2.0 M in THF) cooled to −40° C. in a dry ice/acetonitrile bath. After stirring for 2 h, a solution of methyl 4-formylbenzoate (5.42 g, 33.0 mmol) in THF (50 mL) was added via cannula. The reaction was warmed to 0° C. and stirred for 30 min. Saturated aqueous ammonium chloride was added, the layers were separated and the aqueous layer was extracted with two portions of ethyl acetate. The combined organic extracts were dried (MgSO₄), filtered and concentrated. The residue was purified by flash chromatography (5% MeOH in CH₂Cl₂) to give INTERMEDIATE 5 (8.70 g, 77%). ¹H NMR (CDCl₃) δ_{H} 1.06-1.15 (m, 3H), 1.19-1.28 (m, 3H), 3.19-3.29 (m, 2H), 3.48-3.59 (m, 2H), 3.89-3.92 (s, 3H), 5.88 (d, J=3.07 Hz, 1H), 7.33 (d, J=8.19 Hz, 2H), 7.37 (d, J=7.94 Hz, 2H), 7.46 (d, J=8.19 Hz, 2H), 8.01 (d, J=8.19 Hz, 2H). M.S. (calcd): 342.16 (MH⁺), M.S (found): 342.08 (MH⁺).

### INTERMEDIATE 6: tert-butyl 4-{{4-[(diethylamino)carbonyl]phenyl}[4-(methoxycarbonyl)phenyl]methyl}piperazine-1-carboxylate

To a solution of INTERMEDIATE 5 (8.70 g, 28.7 mmol) in dichloromethane (200 mL) was slowly added thionyl bromide (2.5 mL, 32 mmol) via a dropping funnel. The reaction was stirred for 3 h at room temperature. The solution was washed with saturated aqueous sodium bicarbonate and the organic layer was separated. The aqueous layer was washed with three portions of dichloromethane and the combined organic extracts were dried (MgSO₄), filtered and concentrated.

The crude benzyl bromide was dissolved in acetonitrile (200 mL) and 1-boc-piperazine (5.34 g, 28.7 mmol) was added. After heating the reaction for 2 h at 65° C., the reaction mixture was cooled to room temperature and left to stand overnight. A white precipitate formed, which was filtered to give the product as a colourless solid (12.2 g, 84%). ¹H NMR (CDCl₃) δ_{H} 1.07-1.13 (m, 3H), 1.19-1.25 (m, 3H), 1.44 (s, 9H), 2.28-2.39 (m, 4H), 3.17-3.28 (m, 2H), 3.39-3.47 (m, 4H), 3.48-3.57 (m, 2H), 3.89 (s, 3H), 4.30 (s, 1H), 7.30 (d, J=8.19 Hz, 2H), 7.42 (d, J=8.19 Hz, 2H), 7.49 (d, J=8.19 Hz, 2H), 7.96 (d, J=8.45 Hz, 2H). M.S. (calcd): 510.29 (MH⁺), M.S (found): 510.16 (MH⁺).

### INTERMEDIATE 7: 4-([4-(tert-butoxycarbonyl)piperazin-1-yl]{4-[(diethylamino)carbonyl]phenyl}methyl)benzoic acid

A solution of INTERMEDIATE 6 (11.2 g, 22.0 mmol) and potassium hydroxide (3.7 g, 66 mmol) in a mixture of ethanol (140 mL) and water (60 mL) was heated at 65° C. for 2 h. The solution was cooled to room temperature and concentrated in vacuo. The residue was dissolved in water and the pH was adjusted to ˜7 by adding 1 M HCl. The product crashed out of solution and was filtered using a Buchner funnel. The filtrate was extracted with two portions of ethyl acetate and the combined organic layers were dried (MgSO₄), filtered and concentrated. The residue was combined with the filtered solid to give INTERMEDIATE 7 (8.5 g, 79%) as a colourless solid. ¹H NMR (CDCl₃) δ_{H} 1.05-1.18 (m, 3H), 1.19-1.30 (m, 3H), 1.46 (s, 9H), 2.26-2.45 (m, 4H), 3.18-3.32 (m, 2H), 3.38-3.49 (m, 4H), 3.49-3.61 (m, 2H), 4.32 (s, 1H), 7.32 (d, J=8.19 Hz, 2H), 7.42 (d, J=7.94 Hz, 2H), 7.53 (d, J=8.19 Hz, 2H), 8.02 (d, J=8.19 Hz, 2H). M.S. (calcd): 496.27 (MH⁺), M.S (found): 496.14 (MH⁺).

### INTERMEDIATE 8: tert-butyl 4-({4-[(diethylamino)carbonyl]phenyl}{4-[(methoxycarbonyl)amino]phenyl}-methyl)piperazine-1-carboxylate

INTERMEDIATE 7 (5.3 g, 11 mmol) was dissolved in anhydrous toluene (100 mL) and treated with Et₃N (4.5 mL, 32 mmol). Diphenylphosphoryl azide (4.6 mL, 21 mmol) was added dropwise and the reaction mixture was stirred overnight at room temperature. The solution was heated at reflux for 1.5 h to form the isocyanate and then cooled to room temperature. Anhydrous methanol (100 mL) was added and the reaction was heated at reflux for 4 h. After cooling to room temperature, the reaction mixture was concentrated in vacuo, diluted with ethyl acetate and washed with saturated aqueous sodium bicarbonate. The aqueous phase was washed with two portions of ethyl acetate and the combined organic extracts were dried (MgSO₄), filtered and concentrated. The residue was purified by flash chromatography, eluting with 20% to 80% ethyl acetate in heptane, to give INTERMEDIATE 8 (3.10 g, 55%) as a colourless solid. ¹H NMR (CDCl₃) δ_{H} 1.07-1.14 (m, 3H), 1.19-1.25 (m, 3H), 1.43 (s, 9H), 2.28-2.36 (m, 4H), 3.20-3.30 (m, 2H), 3.37-3.46 (m, 4H), 3.49-3.56 (m, 2H), 3.76 (s, 3H), 4.20 (s, 1H), 7.27-7.34 (m, 6H), 7.41 (d, J=7.94 Hz, 2H). M.S. (calcd): 525.30 (MH⁺), M.S (found): 525.14 (MH⁺).

### INTERMEDIATE 9: 4-[bromo(4-bromophenyl)methyl]-N,N-diethylbenzamide

To a solution of INTERMEDIATE 2 (9.77 g, 27 mmol) in dichloromethane (90 mL) was slowly added thionyl bromide (2.5 mL, 32.4 mmol). The reaction was stirred overnight at room temperature. The solution was washed with saturated aqueous sodium bicarbonate and the organic layer was separated, washed with water, dried (MgSO₄), filtered and concentrated to give 11.5 g of a yellow oil. The product was used directly in the next step.

### INTERMEDIATE 10a: 4-{(4-bromophenyl)[4-(2-fluorobenzyl)piperazin-1-yl]methyl}-N,N-diethylbenzamide

Crude INTERMEDIATE 9 (3.8 g, 9.0 mmol) was dissolved in acetonitrile (90 mL) and 1-(2-fluorobenzyl)piperazine (3.04 g, 15.6 mmol) was added. After heating the reaction overnight at 65° C., the reaction mixture was concentrated and the residue was purified by flash chromatography, eluting with 0% to 100% ethyl acetate in hexanes to give INTERMEDIATE 10a (1.04 g, 21%) as a yellow oil. ¹H NMR (600 MHz, CDCl₃) δ_{H} 1.06-1.15 (m, 3H), 1.20-1.26 (m, 3H), 2.31-2.65 (m, 8H), 3.20-3.29 (m, 2H), 3.49-3.58 (m, 2H), 3.60-3.64 (m, 2H), 4.21-4.23 (m, 1H), 7.01-7.06 (m, 1H), 7.20 (d, J=8.19 Hz, 2H), 7.26-7.31 (m, 4H), 7.33-7.45 (m, 4H). M.S. (calcd): 537.18 (MH⁺), M.S (found): 536.98 (MH⁺).

### INTERMEDIATE 10b: 4-{(4-bromophenyl)[4-(4-fluorobenzyl)piperazin-1-yl]methyl}-N,N-diethylbenzamide

Crude INTERMEDIATE 9 (3.8 g, 9.0 mmol) was dissolved in acetonitrile (90 mL) and 1-(4-fluorobenzyl)piperazine (3.04 g, 15.6 mmol) was added. After heating the reaction for 5 h at 65° C., the reaction mixture was concentrated and the residue was purified by flash chromatography, eluting with 0% to 80% ethyl acetate in hexanes to give INTERMEDIATE 10b (1.94 g, 40%) as a pale yellow solid. ¹H NMR (600 MHz, CDCl₃) δ_{H} 1.04-1.15 (m, 3H), 1.17-1.26 (m, 3H), 2.20-2.64 (m, 8H), 3.19-3.27 (m, 2H), 3.45-3.48 (m, 2H), 3.49-3.56 (m, 2H), 4.20-4.22 (m, 1H), 6.95-7.00 (m, 2H), 7.26 (s, 6H), 7.37 (s, 6H), 7.37 (d, J=7.94 Hz, 2H), 7.40 (d, J=8.19 Hz, 2H). M.S. (calcd): 537.18 (MH⁺), M.S (found): 536.96 (MH⁺).

### COMPOUND 1: (+)-4-[[4-(acetylamino)phenyl](4-benzylpiperazin-1-yl)methyl]-N,N-diethylbenzamide and COMPOUND 2: (−)-4-[[4-(acetylamino)phenyl](4-benzylpiperazin-1-yl)methyl]-N,N-diethylbenzamide

A Smith Process vial was charged with INTERMEDIATE 4 (500 mg, 0.94 mmol), acetamide (67 mg, 1.13 mmol), xantphos (81 mg, 0.14 mmol), Pd₂(dba)₃ (43 mg, 0.047 mmol), cesium carbonate (429 mg, 1.32 mmol) and degassed dioxane (2 mL). The cap was tightened thoroughly and the vessel was exposed to microwave irradiation in a Smith Synthesizer for 2 h at 150° C. The mixture was diluted with ethyl acetate, washed with water and the organic layer was dried (MgSO₄), filtered and concentrated. The residue was purified by reverse phase chromatography using a LUNA C-18 column, gradient 10-50% B in 25 min, flow rate 40 mL/min, 20° C., A: 0.1% TFA in H₂O, B: 0.1% TFA in CH₃CN. The product was obtained as its TFA salt and was lyophilized to give 73 mg (11%) of a colourless solid. The enantiomers were then separated by chiral HPLC using a Chiralpak AD semi-prep column, flow rate 28 mL/min, 5% MeOH/5% EtOH/90% hexanes containing 0.1% diethylamine to give 21.4 mg of COMPOUND 1 and 13.1 mg of COMPOUND 2. The compounds were converted to their TFA salts affording 21.8 mg of COMPOUND 1 and 17.1 mg of COMPOUND 2 as colourless solids.

Analytical Chiral HPLC conditions:

Chiralpak AD, flow rate 1 mL/min

5% MeOH/5% EtOH/90% hexane

COMPOUND 1 retention time: 23.2 minutes

COMPOUND 2 retention time: 29.4 minutes

COMPOUND 1: ¹H NMR free amine (400 MHz, CD₃OD) δ_{H} 1.04-1.15 (m, 3H), 1.16-1.27 (m, 3H), 2.12 (s, 3H), 2.31-2.59 (m, 8H), 3.18-3.29 (m, 2H), 3.46-3.58 (m, 4H), 4.21 (m, 1H), 7.21-7.34 (m, 9H), 7.35-7.43 (m, 4H). M.S. (calcd): 499.3 (MH⁺), M.S (found): 499.0 (MH⁺). HPLC: k′ 4.49; Purity: 94% (215 nm), >99% (254 nm), >99% (280 nm). Conditions: Zorbax C-18, gradient 20-50% B in 25 min, flow rate 1 mL/min, 25° C., A: 0.1% TFA in H₂O, B: 0.1% TFA in CH₃CN; Chiral Purity: >99% (215 nm), >99% (254 nm), >99% (280 nm), Rₜ: 14.9 minutes; Conditions: Chiralcel OD 12% EtOH in 88% hexane containing 0.1% diethylamine. Optical Rotation (TFA salt): [α]¹⁸_{D}=+5.0° (c=0.70, MeOH).

COMPOUND 2: ¹H NMR free amine (400 MHz, CD₃OD) δ_{H} 1.04-1.15 (m, 3H), 1.16-1.27 (m, 3H), 2.12 (s, 3H), 2.31-2.59 (m, 8H), 3.18-3.29 (m, 2H), 3.46-3.58 (m, 4H), 4.21 (m, 1H), 7.21-7.34 (m, 9H), 7.35-7.43 (m, 4H). M.S. (calcd): 499.3 (MH⁺), M.S (found): 499.0 (MH⁺). HPLC: k′ 4.49; Purity: 97% (215 nm), >99% (254 nm), >99% (280 nm). Conditions: Zorbax C-18, gradient 20-50% B in 25 min, flow rate 1 mL/min, 25° C., A: 0.1% TFA in H₂O, B: 0.1% TFA in CH₃CN; Chiral Purity: >99% (215 nm), >99% (254 nm), >99% (280 nm), Rₜ: 17.9 minutes; Conditions: Chiralcel OD 12% EtOH in 88% hexane, containing 0.1% diethylamine. Optical Rotation (TFA salt): [α]¹⁸_{D}=−4.3° (c=0.83, MeOH).

### COMPOUND 3: (−)-Methyl[4-((4-benzylpiperazin-1-yl){4-[(diethylamino)-carbonyl]phenyl}methyl)phenyl]carbamate and COMPOUND 4: (+)-Methyl[4-((4-benzylpiperazin-1-yl){4-[(diethylamino)carbonyl]phenyl}methyl)phenyl]carbamate

Using the same method as for COMPOUND 1 and COMPOUND 2, but replacing acetamide with methyl carbamate (85 mg, 1.13 mmol), provided the racemic product (138 mg, 20%) as a colourless solid and as its TFA salt. The enantiomers were separated by chiral HPLC using a Chiralcel OD semi-prep column, flow rate 18 mL/min, 10% MeOH/10% EtOH/80% hexanes, containing 0.1% diethylamine to give, after treatment with TFA, 88.6 mg of COMPOUND 3 and 64.9 mg of COMPOUND 4 as colourless solids and as their TFA salts.

Analytical Chiral HPLC conditions:

Chiralcel OD, flow rate 1 mL/min

10% MeOH/10% EtOH/80% hexane

COMPOUND 3 retention time: 10.7 minutes

COMPOUND 4 retention time: 15.2 minutes

COMPOUND 3: ¹H NMR free amine (400 MHz, CD₃OD) δ_{H} 1.08 (t, J=6.64 Hz, 3H), 1.22 (t, J=6.64 Hz, 3H), 2.18-2.47 (m, 2H), 2.92-3.11 (m, 2H), 3.19-3.41 (m, 6H), 3.47-3.56 (m, 2H), 3.70 (s, 3H), 4.33 (s, 2H), 4.43 (s, 1H), 7.30-7.41 (m, 6H), 7.48 (s, 5H), 7.54 (d, J=8.20 Hz, 2H). M.S. (calcd): 515.3 (MH⁺), M.S (found): 515.0 (MH⁺). HPLC: k′ 3.76; Purity: >99% (215 nm), >99% (254 nm), >99% (280 nm). Conditions: Zorbax C-18, gradient 10-95% B in 25 min, flow rate 1 mL/min, 25° C., A: 0.1% TFA in H₂O, B: 0.1% TFA in CH₃CN; Chiral Purity: >99% (215 nm), >99% (254 nm), >99% (280 nm), Rₜ: 9.22 minutes; Conditions: Chiralpak AD 30% isopropanol/70% hexane, containing 0.1% DEA. Optical Rotation (TFA salt): [α]^{˜}_{D}=−3.9° (c=1.07, MeOH).

COMPOUND 4: ¹H NMR free amine (400 MHz, CD₃OD) δ_{H} 1.08 (t, J=6.64 Hz, 3H), 1.22 (t, J=6.64 Hz, 3H), 2.18-2.47 (m, 2H), 2.92-3.11 (m, 2H), 3.19-3.41 (m, 6H), 3.47-3.56 (m, 2H), 3.70 (s, 3H), 4.33 (s, 2H), 4.43 (s, 1H), 7.30-7.41 (m, 6H), 7.48 (s, 5H), 7.54 (d, J=8.20 Hz, 2H). M.S. (calcd): 515.3 (MH⁺), M.S (found): 515.0 (MH⁺). HPLC: k′ 3.76; Purity: >99% (215 nm), >99% (254 nm), >99% (280 nm). Conditions: Zorbax C-18, gradient 10-95% B in 25 min, flow rate 1 mL/min, 25° C., A: 0.1% TFA in H₂O, B: 0.1% TFA in CH₃CN; Chiral Purity: >99% (215 nm), >99% (254 nm), >99% (280 nm), Rₜ: 12.81 minutes; Conditions: Chiralpak AD 30% isopropanol/70% hexane, containing 0.1% DEA. Optical Rotation (TFA salt): [α]¹⁸_{D}=+7.9° (c=0.746, MeOH).

### COMPOUND 5: (+)-methyl{4-[{4-[(diethylamino)carbonyl]phenyl}(piperazin-1-yl)methyl]phenyl}carbamate

A solution of INTERMEDIATE 8 (3.00 g, 5.73 mmol) in dry dichloromethane (115 mL) was treated with phenol (10.8 g, 114 mmol) and chlorotrimethylsilane (14.5 mL, 114 mmol). The reaction was stirred for 3 h at room temperature and then concentrated in vacuo. The residue was diluted with dichloromethane and washed with four portions of 2 M NaOH, then water. The organic layer was dried (MgSO₄), filtered and concentrated to give the product (2.11 g, 87%) as a colourless solid. Purity (HPLC-254 nm): >91%. The racemic mixture (2.00 g) was separated by chiral HPLC using a Chiralcel OD semi-prep column, flow rate 18 mL/min, 10% MeOH/10% EtOH/80% hexanes+0.1% diethylamine to give 0.952 g of the (−)-enantiomer and 0.769 g of the (+)-enantiomer as pale yellow solids.

Analytical Chiral HPLC conditions:

Chiralcel OD, flow rate 1 mL/min

10% MeOH/10% EtOH/80% hexane

(−)-ENANTIOMER retention time: 9.26 minutes

(+)-ENANTIOMER retention time: 16.67 minutes

The (+)-enantiomer (150 mg) was repurified by reverse phase chromatography (5% to 45% acetonitrile in water containing 0.1% trifluoroacetic acid). The product was obtained as its trifluoroacetic acid salt and was lyophilized to give COMPOUND 5 (144 mg) as a colourless solid. ¹H NMR (600 MHz, CD₃OD) δ_{H} 1.09 (t, J=6.66 Hz, 3H), 1.22 (t, J=6.66 Hz, 3H), 2.58-2.67 (m, 4H), 3.21-3.27 (m, 6H), 3.52 (q, J=6.40 Hz, 2H), 3.70 (s, 3H), 4.43 (s, 1H), 7.30-7.41 (m, 6H), 7.55 (d, J=8.19 Hz, 2H). M.S. (calcd): 425.3 (MH⁺), M.S (found): 425.0 (MH⁺). HPLC: k′ 2.69; Purity: >94% (215 nm), >99% (254 nm), >99% (280 nm). Conditions: Zorbax C-18, gradient 10-95% B in 25 nm in, flow rate 1 mL/min, 25° C., A: 0.1% TFA in H₂O, B: 0.1% TFA in CH₃CN; Chiral Purity: >99% (215 nm), >99% (254 nm), >99% (280 nm), Rₜ: 16.88 minutes; Conditions: Chiralpak OD 20% ethanol/80% hexane, containing 0.1% DEA. Optical Rotation (TFA salt): [α]¹⁸_{D}=+2.9° (c=1.08, MeOH).

### COMPOUND 6: (+)-Methyl (4-{{4-[(diethylamino)carbonyl]phenyl}[4-(pyridin-2-ylmethyl)piperazin-1-yl]methyl}phenyl)carbamate

A solution of COMPOUND 5 (200 mg, 0.47 mmol) in dry 1,2-dichloroethane (15 mL) was treated with 2-pyridine carboxaldehyde (67 μL, 0.71 mmol) and sodium triacetoxyborohydride (160 mg, 0.75 mmol). The mixture was stirred overnight at room temperature under an atmosphere of nitrogen and then concentrated in vacuo. The residue was purified by reverse phase chromatography (5% to 45% acetonitrile in water containing 0.1% trifluoroacetic acid). The product was obtained as its TFA salt and was lyophilized (H₂O/MeCN) to give COMPOUND 6 as a colourless solid (274 mg, 78%). ¹H NMR (600 MHz, CD₃OD) δ_{H} 1.09 (t, J=6.14 Hz, 3H), 1.22 (t, J=6.91 Hz, 3H), 2.71-2.87 (m, 4H), 3.21-3.27 (m, 2H), 3.33-3.40 (m, 4H), 3.52 (q, J=6.66 Hz, 2H), 3.70 (s, 3H), 4.44 (s, 2H), 4.56 (s, 1H), 7.34 (d, J=8.19 Hz, 2H), 7.36-7.42 (m, 4H), 7.45-7.49 (m, 1H), 7.52 (d, J=7.68 Hz, 1H), 7.57 (d, J=7.94 Hz, 2H), 7.91-7.96 (m, 1H), 8.66 (d, J=4.86 Hz, 1H). M.S. (calcd): 516.3 (MH⁺), M.S (found): 516.0 (MH⁺). HPLC: k′ 2.65; Purity: >99% (215 nm), >99% (254 nm), >99% (280 nm). Conditions: Zorbax C-18, gradient 10-95% B in 25 min, flow rate 1 mL/min, 25° C., A: 0.05% TFA in H₂O, B: 0.05% TFA in CH₃CN; Chiral Purity: >99% (215 nm), >99% (254 nm), >99% (280 nm), Rₜ: 10.45 minutes; Conditions: Chiralpak OD 40% ethanol/60% hexane, containing 0.1% DEA. Optical Rotation (TFA salt): [α]¹⁸_{D}=+7.8° (c=1.02, MeOH).

### COMPOUND 7: (+)-Methyl (4-{{4-[(diethylamino)carbonyl]phenyl}[4-(pyridin-3-ylmethyl)piperazin-1-yl]methyl}phenyl)carbamate

Using the same procedure as COMPOUND 6 with 3-pyridine carboxaldehyde (67 μL, 0.71 mmol) gave COMPOUND 7 (137 mg, 39%) as a colourless solid. ¹H NMR (600 MHz, CD₃OD) δ_{H} 1.09 (t, J=6.66 Hz, 3H), 1.22 (t, J=6.91 Hz, 3H), 2.59-3.02 (m, 4H), 3.12-3.28 (m, 6H), 3.52 (q, J=7.17 Hz, 2H), 3.70 (s, 3H), 4.26 (br s, 2H), 4.78 (s, 1H), 7.34-7.48 (m, 6H), 7.60 (d, J=7.94 Hz, 2H), 7.72-7.80 (m, 1H), 8.19-8.27 (m, 1H), 8.69-8.84 (m, 2H). M.S. (calcd): 516.3 (MH⁺), M.S (found): 516.0 (MH⁺). HPLC: k′ 2.45; Purity: >99% (215 nm), >99% (254 nm), >99% (280 nm). Conditions: Zorbax C-18, gradient 10-95% B in 25 min, flow rate 1 mL/min, 25° C., A: 0.05% TFA in H₂O, B: 0.05% TFA in CH₃CN; Chiral Purity: >99% (215 nm), >99% (254 nm), >99% (280 nm), Rₜ: 9.38 minutes, Conditions: Chiralpak OD 40% ethanol/60% hexane, containing 0.1% DEA. Optical Rotation (TFA salt): [α]¹⁸_{D}=+8.2° (c=0.61, MeOH).

### COMPOUND 8: (+)-Methyl (4-{{4-[(diethylamino)carbonyl]phenyl}[4-(pyridin-4-ylmethyl)piperazin-1-yl]methyl}phenyl)carbamate

Using the same procedure as COMPOUND 6 with 4-pyridine carboxaldehyde (68 μL, 0.71 mmol) gave COMPOUND 8 (297 mg, 85%) as a colourless solid. ¹H NMR (600 MHz, CD₃OD) δ_{H} 1.09 (t, J=6.91 Hz, 3H), 1.23 (t, J=6.66 Hz, 3H), 3.30 (m, 8H), 3.24 (q, J=7.17 Hz, 2H), 3.53 (q, J=7.17 Hz, 2H), 3.71 (s, 3H), 4.05-4.16 (br s, 2H), 5.04-5.30 (br s, 1H), 7.44 (d, J=7.68 Hz, 2H), 7.48-7.54 (m, 4H), 7.68 (d, J=7.68 Hz, 2H), 7.90-7.97 (m, 2H), 8.76 (d, J=5.89 Hz, 2H). M.S. (calcd): 516.3 (MH⁺), M.S (found): 516.0 (MH⁺). HPLC: k′ 2.32; Purity: >99% (215 nm), >99% (254 nm), >99% (280 nm). Conditions: Zorbax C-18, gradient 10-95% B in 25 min, flow rate 1 mL/min, 25° C., A: 0.05% TFA in H₂O, B: 0.05% TFA in CH₃CN; Chiral Purity: >99% (215 nm), >99% (254 nm), >99% (280 nm), Rₜ: 13.02 minutes; Conditions: Chiralpak OD 40% ethanol/60% hexane, containing 0.1% DEA. Optical Rotation (TFA salt): [α]¹⁸_{D}=+9.5° (c=1.12, MeOH).

### COMPOUND 9: (+)-4-{[4-(acetylamino)phenyl][4-(2-fluorobenzyl)piperazin-1-yl]methyl}-N,N-diethylbenzamide and COMPOUND 10: (−)-4-{[4-(acetylamino)phenyl][4-(2-fluorobenzyl)piperazin-1-yl]methyl}-N,N-diethylbenzamide

Two Smith Process vials were each charged with INTERMEDIATE 10a (0.53 g, 0.99 mmol), acetamide (70 mg, 1.1 mmol), xantphos (86 mg, 0.15 mmol), Pd-₂(dba)₃ (45 mg, 0.05 mmol), cesium carbonate (1.61 g, 4.95 mmol) and dioxane (2.5 mL). The caps were tightened thoroughly and the vessels were exposed to microwave irradiation in a Smith Synthesizer for 2 h at 150° C. The combined vials were diluted with ethyl acetate, washed with water and the organic layer was dried (MgSO₄), filtered and concentrated. The residue was purified by reverse phase chromatography using a LUNA C-18 column, 250×50 mm, gradient 20-60% B in 25 min, flow rate 50 mL/min, 20° C., A: 0.1% TFA in H₂O, B: 0.1% TFA in CH₃CN. The product was obtained as its TFA salt and was lyophilized to give 268 mg (18%) of a colourless solid. The enantiomers were then separated by chiral HPLC using a Chiralpak AD semi-prep column, flow rate 28 mL/min, 5% MeOH/5% EtOH/90% hexanes+0.1% diethylamine. The enantiomers were converted to their HCl salts affording 30 mg of COMPOUND 9 and 20 mg of COMPOUND 10 as colourless solids.

Analytical Chiral HPLC conditions:

Chiralpak AD, flow rate 1 mL/min

5% MeOH/5% EtOH/90% hexane

COMPOUND 9 retention time: 20.69 minutes

COMPOUND 10 retention time: 25.07 minutes

COMPOUND 9: ¹H NMR (400 MHz, CD₃OD) δ_{H} 1.09 (t, J=6.64 Hz, 3H), 1.22 (t, J=6.84 Hz, 3H), 2.09 (s, 3H), 2.90-3.17 (m, 4H), 3.20-3.28 (m, 2H), 3.48-3.64 (m, 6H), 4.50 (s, 2H), 4.92-5.16 (br s, 1H), 7.24-7.34 (m, 2H), 7.40 (d, J=8.20 Hz, 2H), 7.52-7.64 (m, 6H), 7.71-7.77 (m, 2H). M.S. (calcd): 517.3 (MH⁺), M.S (found): 517.0 (MH⁺). HPLC: k′ 3.31; Purity: >99% (215 nm), >99% (254 nm), >99% (280 nm). Conditions: Zorbax C-18, gradient 10-95% B in 25 min, flow rate 1 mL/min, 25° C., A: 0.1% TFA in H₂O, B: 0.1% TFA in CH₃CN; Chiral Purity: >99% (215 nm), 98% (254 nm), 98% (280 nm), Rₜ: 19.99 minutes; Conditions: Chiralpak AD 5% EtOH/5% MeOH/90% hexane, containing 0.1% DEA. Optical Rotation: [α]¹⁷_{D}=+7.8° (c=0.58, MeOH).

COMPOUND 10: ¹H NMR (400 MHz, CD₃OD) δ_{H} 1.09 (t, J=6.64 Hz, 3H), 1.22 (t, J=6.84 Hz, 3H), 2.09 (s, 3H), 2.90-3.17 (m, 4H), 3.20-3.28 (m, 2H), 3.48-3.64 (m, 6H), 4.50 (s, 2H), 4.92-5.16 (br s, 1H), 7.24-7.34 (m, 2H), 7.40 (d, J=8.20 Hz, 2H), 7.52-7.64 (m, 6H), 7.71-7.77 (m, 2H). M.S. (calcd): 517.3 (MH⁺), M.S (found): 517.0 (MH⁺). HPLC: k′ 3.31; Purity: >99% (215 nm), >99% (254 nm), >99% (280 nm). Conditions: Zorbax C-18, gradient 10-95% B in 25 min, flow rate 1 mL/min, 25° C., A: 0.1% TFA in H₂O, B: 0.1% TFA in CH₃CN; Chiral Purity: >99% (215 nm), 93% (254 nm), 93% (280 nm), Rₜ: 24.32 minutes; Conditions: Chiralpak AD 5% EtOH/5% MeOH/90% hexane, containing 0.1% DEA. Optical Rotation: [α]¹⁷_{D}=−8.4° (c=0.45, MeOH).

### COMPOUND 11: (+)-4-{[4-(acetylamino)phenyl][4-(4-fluorobenzyl)piperazin-1-yl]methyl}-N,N-diethylbenzamide and COMPOUND 12: (−)-4-{[4-(acetylamino)phenyl][4-(4-fluorobenzyl)piperazin-1-yl]methyl}-N,N-diethylbenzamide

Three Smith Process vials were each charged with INTERMEDIATE 10b (0.50 g, 0.93 mmol), acetamide (66 mg, 1.2 mmol), xantphos (81 mg, 0.14 mmol), Pd-₂(dba)₃ (43 mg, 0.05 mmol), cesium carbonate (1.51 g, 4.65 mmol) and dioxane (2.5 mL). The caps were tightened thoroughly and the vessels were exposed to microwave irradiation in a Smith Synthesizer for 2 h at 150° C. The combined vials were diluted with ethyl acetate, washed with water and the organic layer was dried (MgSO₄), filtered and concentrated. The residue was purified by reverse phase chromatography using a LUNA C-18 column, 250×50 mm, gradient 15-65% B in 25 min, flow rate 50 mL/min, 20° C., A: 0.1% TFA in H₂O, B: 0.1% TFA in CH₃CN. The product was obtained as its TFA salt and was lyophilized to give 750 mg (32%) of a colourless solid. The enantiomers were then separated by chiral HPLC using a Chiralpak AD semi-prep column, flow rate 28 mL/min, 5% MeOH/5% EtOH/90% hexanes containing 0.1% diethylamine. The enantiomers were converted to their HCl salts affording 133 mg of COMPOUND 11 and 90 mg of COMPOUND 12 as colourless solids.

Analytical Chiral HPLC conditions:

Chiralpak AD, flow rate 1 mL/min

5% MeOH/5% EtOH/90% hexane

COMPOUND 11 retention time: 21.99 minutes

COMPOUND 12 retention time: 27.82 minutes

COMPOUND 11: ¹H NMR (400 MHz, CD₃OD) δ_{H} 1.06 (t, J=7.03 Hz, 3H), 1.19 (t, J=6.64 Hz, 3H), 2.05 (s, 3H), 2.87-3.14 (m, 4H), 3.17-3.25 (m, 2H), 3.41-3.53 (m, 6H), 4.35-4.38 (m, 2H), 4.90 (br s, 1H), 7.16-7.22 (m, 2H), 7.35 (d, J=8.20 Hz, 2H), 7.47-7.57 (m, 6H), 7.63-7.70 (m, 2H). M.S. (calcd): 517.3 (MH⁺), M.S (found): 517.0 (MH⁺). HPLC: k′ 3.39; Purity: >99% (215 nm), >99% (254 nm), >99% (280 nm). Conditions: Zorbax C-18, gradient 10-95% B in 25 min, flow rate 1 mL/min, 25° C., A: 0.1% TFA in H₂O, B: 0.1% TFA in CH₃CN; Chiral Purity: >99% (215 nm), >99% (254 nm), >99% (280 nm), Rₜ: 17.53 minutes; Conditions: Chiralcel OD 10% EtOH/90% hexane, containing 0.1% DEA. Optical Rotation: [α]¹⁷_{D}=+11.1° (c=0.69, MeOH).

COMPOUND 12: ¹H NMR (400 MHz, CD₃OD) δ_{H} 1.06 (t, J=7.03 Hz, 3H), 1.19 (t, J=6.64 Hz, 3H), 2.05 (s, 3H), 2.87-3.14 (m, 4H), 3.17-3.25 (m, 2H), 3.41-3.53 (m, 6H), 4.35-4.38 (m, 2H), 4.90 (br s, 1H), 7.16-7.22 (m, 2H), 7.35 (d, J=8.20 Hz, 2H), 7.47-7.57 (m, 6H), 7.63-7.70 (m, 2H). M.S. (calcd): 517.3 (MH⁺), M.S (found): 517.0 (MH⁺). HPLC: k′ 3.41; Purity: >99% (215 nm), >99% (254 nm), >99% (280 nm). Conditions: Zorbax C-18, gradient 10-95% B in 25 min, flow rate 1 mL/min, 25° C., A: 0.1% TFA in H₂O, B: 0.1% TFA in CH₃CN; Chiral Purity: >99% (215 nm), >99% (254 nm), >99% (280 nm), Rₜ: 20.53 minutes; Conditions: Chiralcel OD 10% EtOH/90% hexane, containing 0.1% DEA. Optical Rotation: [α]¹⁷_{D}=−11.5° (c=0.65, MeOH).

1. A compound of formula I, a pharmaceutically acceptable salt thereof, diastereomers, enantiomers, or mixtures thereof: 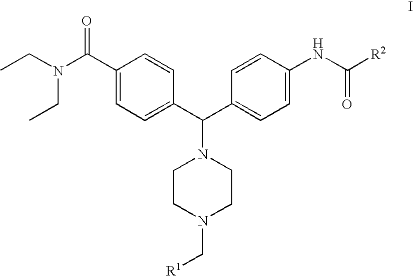
wherein
R¹ is phenyl or C₃₋₅heteroaryl, wherein said phenyl and C₃₋₅heteroaryl are optionally substituted with one or more groups selected from C₁₋₄alkyl, C₁₋₄alkoxy, halogen, amino, —CF₃, and C₁₋₄acyl; and R² is —H, C₁₋₄alkyl, or C₁₋₄alkoxy.

2. A compound according to claim 1, wherein
R¹ is phenyl, pyridyl, furyl, thienyl, imidazolyl, triazolyl, pyrrolyl, thiazolyl, or pyridyl-N-oxide, wherein said phenyl, pyridyl, furyl, thienyl, imidazolyl, triazolyl, pyrrolyl, thiazolyl, and pyridyl-N-oxide are optionally substituted with one or more groups selected from halogen and C₁₋₄alkyl; and
R² is —H, methyl, ethyl, methoxy, or ethoxy.

3. A compound according to claim 1, wherein
R¹ is phenyl or pyridyl, wherein said phenyl and pyridyl are optionally substituted with one or more groups selected from methyl and fluoro; and
R² is methyl or methoxy.

4. A compound according to claim 1, wherein
R¹ is phenyl, 2-fluorophenyl, 4-fluorophenyl, 2-pyridyl, 3-pyridyl, or 4-pyridyl; and
R² is methyl or methoxy.

5. A compound according to claim 1, wherein the compound is selected from:
(+)-4-[[4-(acetylamino)phenyl](4-benzylpiperazin-1-yl)methyl]-N,N-diethylbenzamide;
(−)-4-[[4-(acetylamino)phenyl](4-benzylpiperazin-1-yl)methyl]-N,N-diethylbenzamide;
(−)-Methyl[4-((4-benzylpiperazin-1-yl){4-[(diethylamino)-carbonyl]phenyl}methyl)phenyl]carbamate;
(+)-Methyl[4-((4-benzylpiperazin-1-yl){4-[(diethylamino)carbonyl]phenyl}methyl)phenyl]carbamate;
(+)-Methyl (4-{{4-[(diethylamino)carbonyl]phenyl}[4-(pyridin-2-ylmethyl)piperazin-1-yl]methyl}phenyl)carbamate;
(+)-Methyl (4-{{4-[(diethylamino)carbonyl]phenyl}[4-(pyridin-3-ylmethyl)piperazin-1-yl]methyl}phenyl)carbamate;
(+)-Methyl (4-{{4-[(diethylamino)carbonyl]phenyl}[4-(pyridin-4-ylmethyl)piperazin-1-yl]methyl}phenyl)carbamate;
(+)-4-{[4-(acetylamino)phenyl][4-(2-fluorobenzyl)piperazin-1-yl]methyl}-N,N-diethylbenzamide;
(−)-4-{[4-(acetylamino)phenyl][4-(2-fluorobenzyl)piperazin-1-yl]methyl}-N,N-diethylbenzamide;
(+)-4-{[4-(acetylamino)phenyl][4-(4-fluorobenzyl)piperazin-1-yl]methyl}-N,N-diethylbenzamide;
(−)-4-{[4-(acetylamino)phenyl][4-(4-fluorobenzyl)piperazin-1-yl]methyl}-N,N-diethylbenzamide;
and pharmaceutically acceptable salts thereof.

6. A pharmaceutical composition comprising a compound according to claim 1 and a pharmaceutically acceptable carrier.

7. A pharmaceutical composition comprising a compound according to claim 5 and a pharmaceutically acceptable carrier.

8. A method for the therapy of pain in a warm-blooded animal, comprising administering to said animal in need of such therapy a therapeutically effective amount of a compound according to claim 1.

9. A method for the therapy of pain in a warm-blooded animal, comprising administering to said animal in need of such therapy a therapeutically effective amount of a compound according to claim 5.

10. A method for the therapy of depression in a warm-blooded animal, comprising administering to said animal in need of such therapy a therapeutically effective amount of a compound according to claim 1.

11. A process for preparing a compound of formula I, comprising: 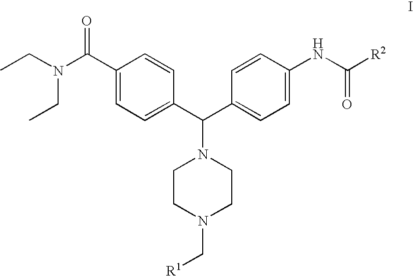
reacting a compound of formula II with R²—C(═O)—NH₂, 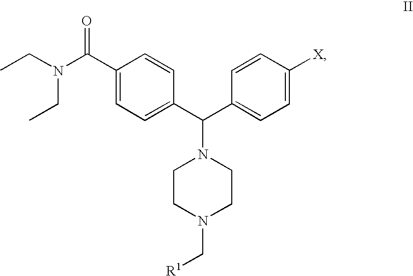
wherein
X is halogen or —OTf;
R¹ is phenyl or C₃₋₅heteroaryl, wherein said phenyl and C₃₋₅heteroaryl are optionally substituted with one or more groups selected from C₁₋₄alkyl, C₁₋₄alkoxy, halogen, amino, —CF₃, and C₁₋₄acyl; and
R² is —H, C₁₋₄alkyl, or C₁₋₄alkoxy.

12. A process for preparing a compound of formula I, comprising: 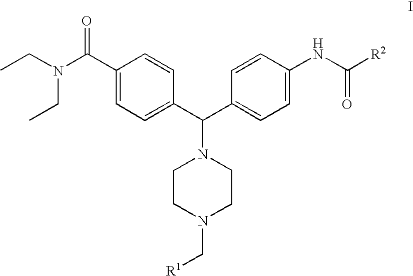
reacting a compound of formula III with R¹—CHO or R¹—CH₂X: 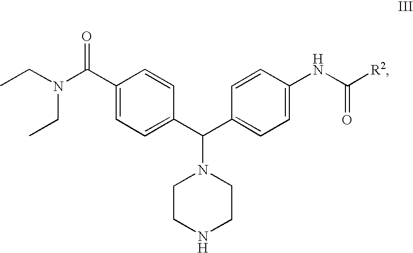
wherein
X is halogen or —OTf;
R¹ is phenyl or C₃₋₅heteroaryl, wherein said phenyl and C₃₋₅heteroaryl are optionally substituted with one or more groups selected from C₁₋₄alkyl, C₁₋₄alkoxy, halogen, amino, —CF₃, and C₁₋₄acyl; and
R² is —H, C₁₋₄alkyl, or C₁₋₄alkoxy.

13. A compound selected from methyl {4-[{4-[(diethylamino)carbonyl]phenyl}(piperazin-1-yl)methyl]phenyl}carbamate and pharmaceutically acceptable salts thereof.

14. A method for the therapy of depression in a warm-blooded animal, comprising administering to said animal in need of such therapy a therapeutically effective amount of a compound according to claim 5.
